(19) United States
(12) Patent Application Publication
(10) Pub. No.: US 2026/0324693 A1
(43) Pub. Date: Oct. 22, 2026

(54) Implanting Device Using Multidimensional Wave Frequency Scalar Wave-Energy Information

(71) Applicants: Xianfa Meng, Flushing, NY (US); Angella Lu, Flushing, NY (US); Jiapeng Hao, Oakland Gardens, NY (US); Qingxi Meng, FLUSHING, NY (US); XILIANG LIU, Flushing, NY (US)

(72) Inventors: Xianfa Meng, Flushing, NY (US); Angella Lu, Flushing, NY (US); Jiapeng Hao, Oakland Gardens, NY (US); Qingxi Meng, FLUSHING, NY (US); XILIANG LIU, Flushing, NY (US)

(21) Appl. No.: 19/104,649

(22) PCT Filed: Aug. 16, 2023
(86) PCT No.: PCT/US23/30407

§ 371 Date: Feb. 18, 2025

Related U.S. Application Data
(60) Provisional application No. 63/398,365, filed on Aug. 16, 2022.

(51) Int. Cl. A23L 5/30; H01F 38/20; H04B 13/00; H05K 7/20

(52) U.S. Cl. CPC A23L 5/30; H01F 38/20; H04B 13/00; H05K 7/20145; H05K 7/20154

(57) ABSTRACT

Disclosed is an implanting device using multidimensional wave frequency scalar wave-energy information, which includes an elongated cabin, a main control panel, a hatch door, a scalar-wave generating device, at least one Tesla coil, a processing system, and a power supply circuit.

The current application is a 371 of international Patent Cooperation Treaty (PCT) application PCT/US2023/030407 filed on Aug. 16, 2023. The PCT application PCT/US2023/030407 claims a priority to the U.S. provisional patent application Ser. No. 63/398,365 filed on Aug. 16, 2022.

### FIELD OF THE INVENTION

The present invention generally relates a scalar wave energy field generating device. More specifically, the present invention can be used to improve the quality of various industrial articles, agricultural products, and food products.

### BACKGROUND OF THE INVENTION

Scalar waves, as a relatively new concept, were first introduced by Nicholas Tesla more than 100 years ago. It was discovered by Tesla and is therefore commonly known as a Tesla wave. The study of scalar waves is inseparable from the consideration of quantum technology. The research significance of scalar wave quantum technology and the wide application field are extraordinary. Through the understanding of foreign-related technologies and the research of this technology and the collection of relevant information, it is summarized as follows:
1. A scalar wave is different from an ordinary electromagnetic wave. Ordinary electromagnetic waves are transverse waves, also known as Hertz waves. When encountering obstacles such as water or gold, ordinary electromagnetic waves are either absorbed or reflected, and their energy decays rapidly with the increase of distance. Scalar waves, on the other hand, are longitudinal waves that do not decay with distance.
2. The speed of the scalar wave is faster than the speed of light, in which it can instantly penetrate any shielding, even through a Faraday screen.
3. Scalar waves can also transmit energy, and in the transmission process, the energy can be amplified several times or even dozens of times.
4. Scalar energy is similar to the Psi (gas) energy emitted by the human body. Scalar energy can be transmitted through metal and silk or can be stored in water, wood, stone, and related metal materials.
5. Scalar energy has negative entropy (energy and information get more orderly transfer) properties, etc. It has been proven through experimentation that scalar energy is non-linear and non-hertzian. This form of energy has the ability to carry information, which can transmit energy and information over a long distance with very little attenuation because they are in a resonance state.

Through market research and the application practice of quantum health technology in the past five years, the present invention implements scalar wave technology, which is a new technology with revolutionary significance. Utilizing a unique scalar wave frequency generated by the present invention to form a multi-dimensional energy field, through the principle of wave frequency resonance, the multi-dimensional energy field forms a resonance effect with the items in the cabin body. The multi-dimensional energy field can then rapidly improve the energy value of goods or food and the work effect of drugs and health products.

The properties of the multi-dimensional wave frequency energy information implantation in improving and enhancing the quality of goods can reduce the surface tension of a product, which can then be more easily absorbed by the human body. Importantly, a product processed by multi-dimensional wave frequency energy information allows the efficacy of the product to be more easily transferred to the cell so that a charge generated across the cell membrane reaches the best level, is easier to enter cell, regulates the body energy field balance. Consequently, the whole body energy then increases, which accelerates the body metabolism and improves the body function.

### SUMMARY OF THE INVENTION

The present invention adopts the international standard for scalar wave full spectrum technology [i.e., a consolidated annotated frequency list (CAFL)]. The present invention also uses the quantum resonance system (QRS) software program control system to form a multidimensional scalar wave energy field through the scalar wave frequency generated by a specific coil device. The present invention also takes advantage of the characteristics of the scalar wave to enlarge energy, carry information, and penetrate all objects. The principle of wave frequency resonance quickly activates the energy state of the goods in a cabin of the present invention and improves the quality of the goods in the cabin of the present invention.

Resonance is the key technology to produce scalar waves, and objects in a resonance state produce high-intensity energy [i.e., resonance here is not traditional inductance-capacitance (LC) electromagnetic resonance]. At low power, scalar wave devices are prone to the energy multiplier effect, wherein the output energy is greater than the input energy. The present invention transmits energy pulse signal and frequency signal through a computer software and establishes scalar wave energy field through a specific quantum convert (QC) coil. The technique of scalar wave resonance is established, and the problem of scalar energy field with directional specific frequency or energy information is mainly solved.

An objective of the present invention is to provide an application and practice of scalar wave quantum technology in the environment, energy, biomedicine, and other fields. Another objective of present invention is to provide wireless power transmission via scalar waves. Another objective of the present invention is to verify theory related to scalar waves and conduct experiments related to scalar waves. Another objective of the present invention is to implement bio-physical interactions of scalar energy on cellular mechanisms of living life.

### BRIEF DESCRIPTION OF THE DRAWINGS

FIG. 1 is a front perspective view of an exemplary embodiment for the present invention, wherein the exemplary embodiment is shown as a multidimensional wave frequency energy information with an implanting compartment.

FIG. 2 is a front view of the exemplary embodiment for the present invention, wherein the exemplary embodiment is shown as the multi-dimensional wave frequency energy information with the implanting compartment and a control panel.

FIG. 3 is a rear perspective view of the exemplary embodiment for the present invention, wherein the exemplary embodiment is shown as the multidimensional wave frequency energy information with the implanting compartment.

FIG. 4 is a block diagram showing the structure of the multi-dimensional wave frequency energy information with an implanting compartment.

FIG. 5 is a schematic illustration of the winding and working principle of the multi-dimensional wave frequency energy information with an implanting compartment.

FIG. 6 is a front perspective view of an exemplary embodiment for the present invention, wherein the exemplary embodiment is shown with a large rectangular-shaped shell.

FIG. 7 is a process diagram of the multi-dimensional wave frequency energy information implanting.

FIG. 8 is a schematic illustration of a multi-dimensional wave frequency energy information device with an implanting compartment.

FIG. 9 is a schematic cross-sectional illustration of the structure of the multi-dimensional wave frequency energy information device with the implanting compartment.

FIG. 10 is a decomposition schematic illustration of the multi-dimensional wave frequency energy information device with the implanting compartment.

### DETAILED DESCRIPTION OF THE INVENTION

All illustrations of the drawings are for the purpose of describing selected versions of the present invention and are not intended to limit the scope of the present invention.

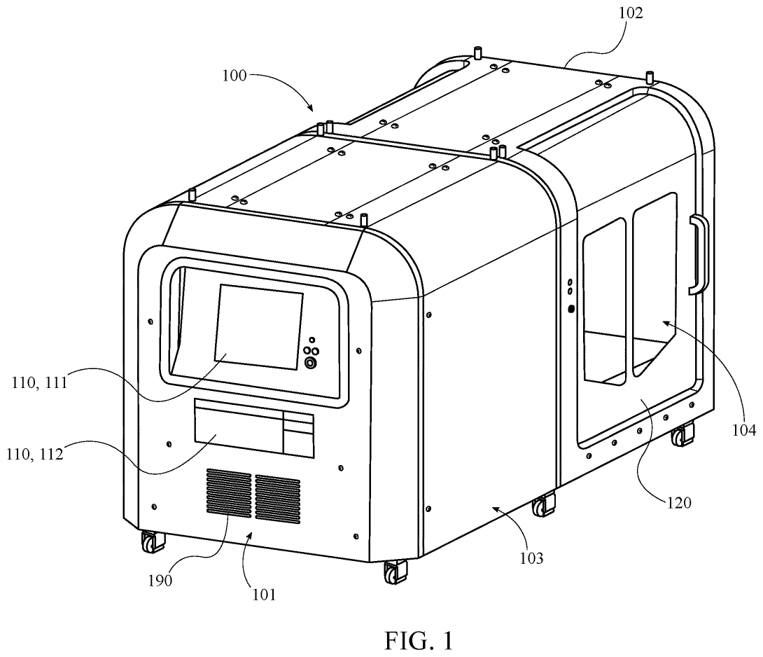

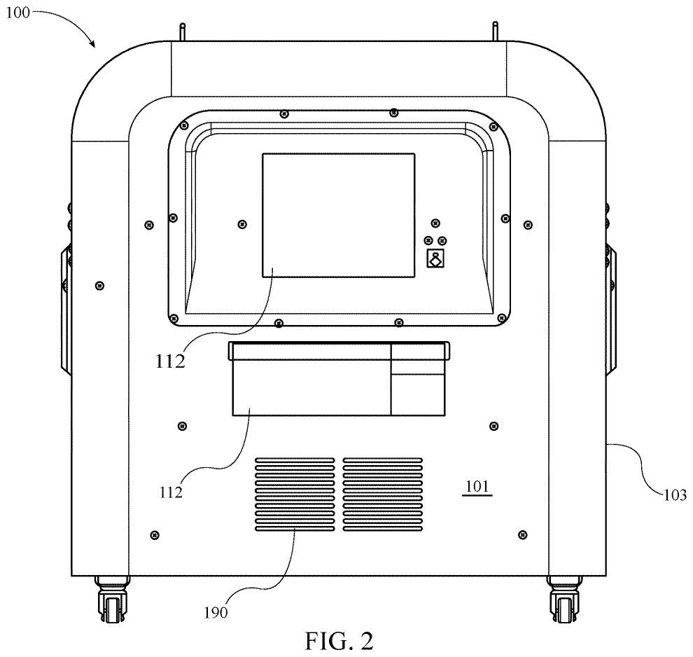

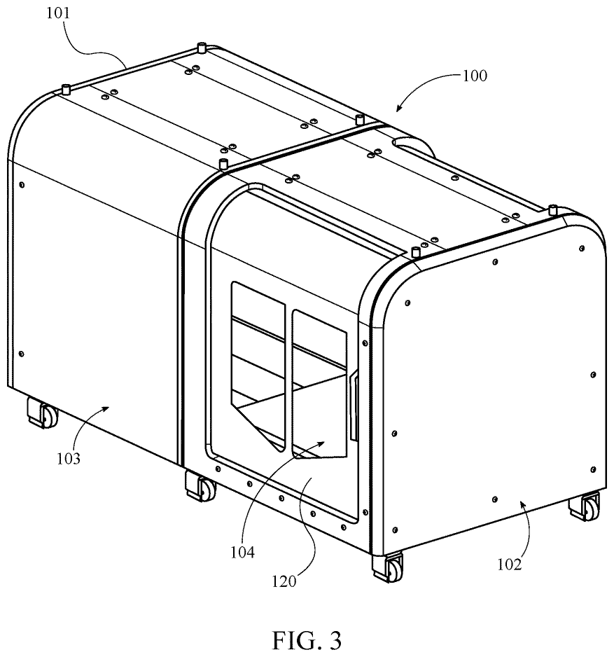

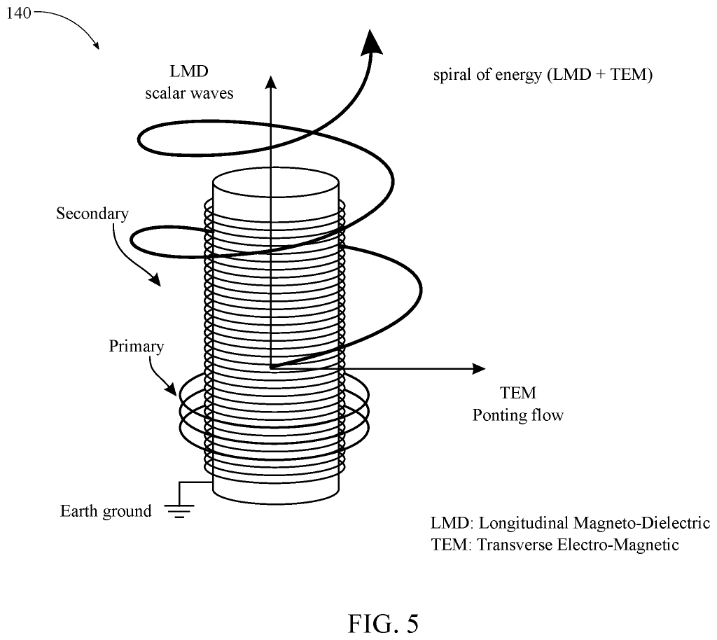

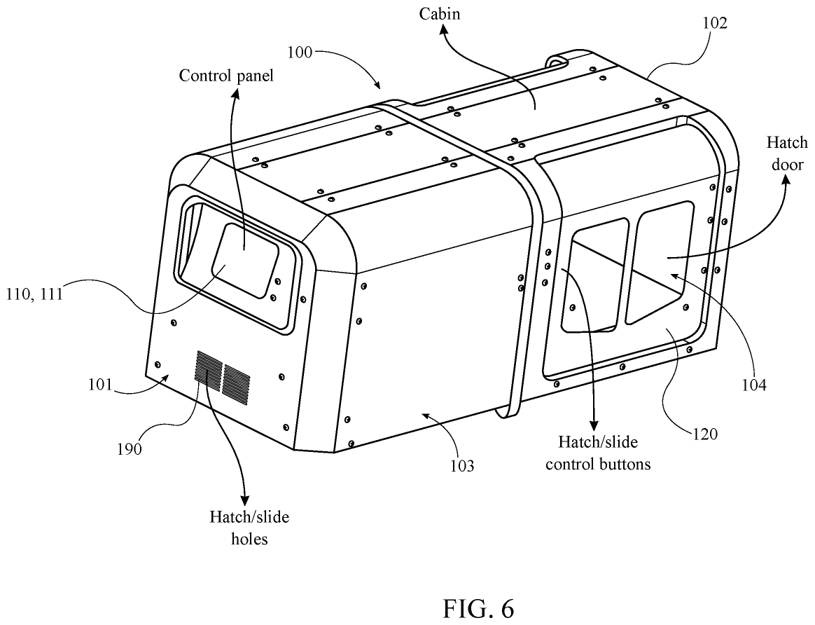

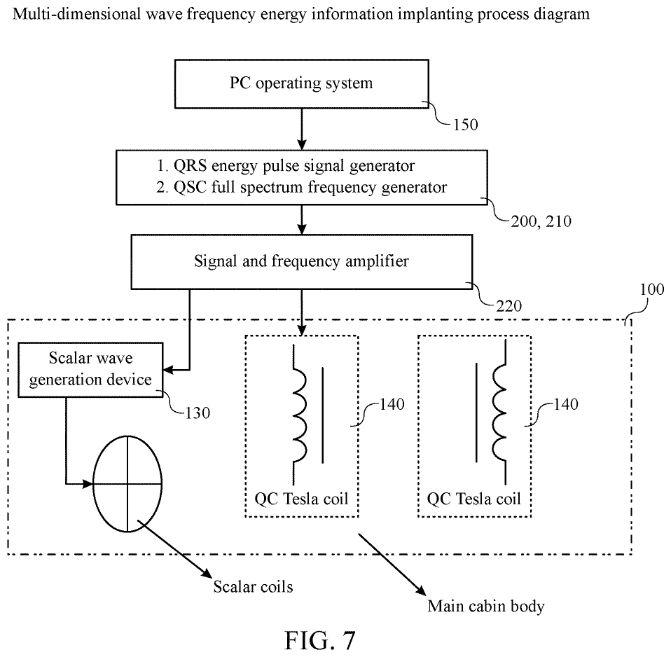

As can be seen in FIGS. 1 through 7, the present invention is an implanting device using multidimensional wave frequency scalar wave-energy information, which can be used for a variety of utilities. The main utility of the present invention is to determine the age of an artifact that is inserted and analyzed by the present invention. The present invention comprises an elongated cabin 100, a main control panel 110, a hatch door 120, a scalar-wave generating device 130, at least one Tesla coil 140, a processing system 150, and a power supply circuit 160. The elongated cabin 100 is the main housing of the present invention and is used as a base for all of the other components of the present invention to be connected together. The main control panel 110 allows a user to input electronic commands or digital information into the present invention and allows the user to view electronic outputs or digital information from the present invention. The scalar-wave generating device 130 is used to form a multi-dimensional energy field, which creates a resonance effect on an item inside the elongated cabin 100. The at least one Tesla coil 140 is used to form zero-point energy vortices, which produces a special harmonic interference and a clockwise rotation of the energy field, and this harmonic energy is several times stronger than the original. The processing system 150 is used to receive, store, and analyze information for the present invention and is used to receive, process, and execute commands for the present invention. The processing system 150 can be, but is not limited to an integrated chipset, an integrated microcontroller, a personal computing device (e.g., a smartphone, a desktop, a laptop, a tablet personal computer, etc.) that is communicably coupled to the rest of the present invention, or any other kind of computing device. The power supply circuit 160 provides electrical power to all of the electrical and electronic components of the present invention. The power supply circuit 160 can provide electrical power from a portable power source (i.e., a battery, a gasoline-powered generator, etc.) or a stationary power source (i.e., an electrical outlet).

As can be seen in FIGS. 1 through 7, the general configuration of the aforementioned components allows the present invention to effectively and efficiently make use of multidimensional wave frequency scalar wave-energy information. The elongated cabin 100 comprises comprising a first cabin end 101, a second cabin end 102, a lateral cabin portion 103, and a hatchway 104. The first cabin end 101 and the second cabin end 102 are the two opposing ends of the elongated cabin 100, and the lateral cabin portion 103 is a tubular body that is positioned in between the first cabin end 101 and the second cabin end 102. The hatchway 104 is an opening that traverses into the elongated cabin 100 and is thus integrated into the lateral cabin portion 103, adjacent to the second cabin end 102. The hatch door 120 is mounted across the hatchway 104 so that the hatchway 104 can be closed off during the operation of the present invention. In addition, the main control panel 110 is externally mounted to the first cabin end 101, which allows a user of the present invention to easily identify and interact with the main control panel 110. Moreover, the scalar-wave generating device 130 and the at least one Tesla coil 140 being mounted within the elongated cabin 100 so that the scalar-wave generating device 130 and the at least one Tesla coil 140 are able to interact with an item placed inside of the elongated cabin 100. The scalar-wave generating device 130 is positioned adjacent to the first cabin end 101, while the at least one Tesla coil 140 is positioned offset from the first cabin end 101, which prevents the scalar-wave generating device 130 and the at least one Tesla coil 140 from functionally interfering with each other. The main control panel 110, the scalar-wave generating device 130, and the at least one Tesla coil 140 are electronically connected to the processing system 150 so that the processing system 150 can act as a hub to exchange and process information and commands amongst these electronic components of the present invention. The control panel, the scalar-wave generating device 130, the at least one Tesla coil 140, and the processing system 150 are electrically connected to the power supply circuit 160, which allows the power supply circuit 160 to readily provide electrical power to the main control panel 110, the scalar-wave generating device 130, the at least one Tesla coil 140, and the processing system 150.

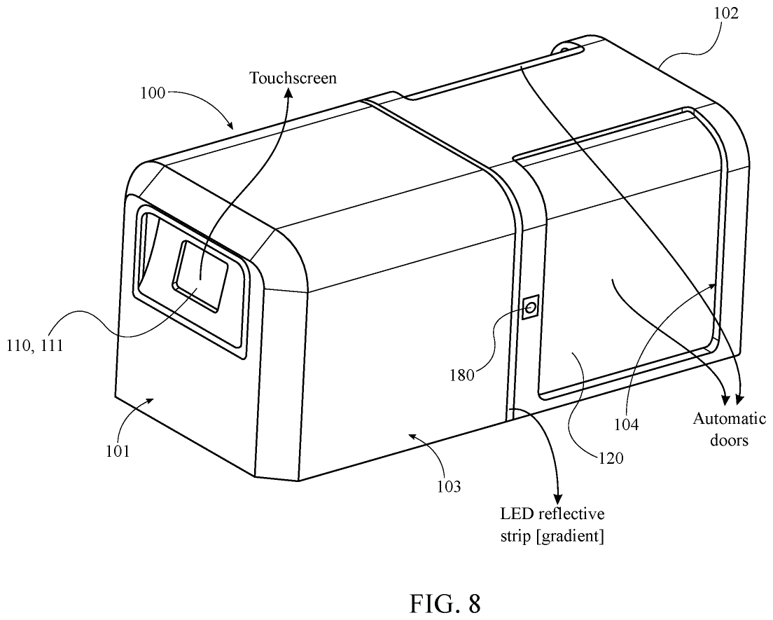

As can be seen in FIGS. 8 through 10, the present invention may further comprise an automatic door-sliding mechanism 170 and an electronic door control 180. The automatic door-sliding mechanism 170 is used to slide the hatch door 120 from a position of closing off the hatchway 104 to a position of opening up the hatchway 104 and vice versa. Thus, the hatch door 120 is operatively mounted to the hatchway 104 by the automatic door-sliding mechanism 170, wherein the automatic door-sliding mechanism 170 is used to open and close the hatch door 120 in order to provide internal access to the elongated cabin 100 through the hatchway 104. In addition, the electronic door control 180 allows a user to actuate the operation of the automatic door-sliding mechanism 170. Thus, the electronic door control 180 is electronically connected to the automatic door-sliding mechanism 170 in order to communicate an actuation of electronic door control 180 to the automatic door-sliding mechanism 170. The electronic door control 180 is also mounted external to the lateral cabin portion 103, adjacent to the hatchway 104, so that a user would instantly recognize the electronic door control 180 and its functionality. Moreover, the automatic door-sliding mechanism 170 and the electronic door control 180 is electrically connected to the power supply circuit 160, which allows the power supply circuit 160 to readily provide electrical power to, the automatic door-sliding mechanism 170 and the electronic door control 180.

As can be seen in FIGS. 1, 2, 6, and 8 through 10, the main control panel 110 may comprise a display screen 111 and at least one control button 112. The display screen 111 allows for visual outputs to be seen by a user through the main control panel 110, while the at least one control button 112 allows a user to enter an input through the main control panel 110. In some embodiments, the display screen 111 can be a touchscreen with or without the at least one control button 112. The at least control button 112 can include, but is not limited to, a power switch, a quantum resonance system (QRS) control switch, and a quantum scalar convert (QSC) control switch. Moreover, the display screen 111 is positioned offset from a base of the lateral cabin portion 103 so that the display screen 111 is in an optimal position to be viewed by a user. The at least one control button 112 is peripherally positioned around the display screen 111 so that a user can easily enter an input based on what the user has seen on the display screen 111.

As can be seen in FIGS. 1, 2, and 6, the present invention may further comprise a heat-sinking ventilation mechanism 190, which is used to collect heat inside of the elongated cabin 100 and to then exhaust the heat out of the elongated cabin 100. The high-sinking ventilation mechanism is preferably a heat sink in thermal communication with an interior of the elongated cabin 100, wherein the heat sink is positioned adjacent to a vent of the elongated cabin 100 and is being blown upon by a fan in a direction through the vent and out of the elongated cabin 100. Thus, the heat-sinking ventilation mechanism 190 being integrated into the first cabin end 101 and is positioned in between the main control panel 110 and a base of the lateral cabin portion 103, which prevents the heat-sinking ventilation mechanism 190 from functionally interfering with the other components of the present invention while still being able to remove heat from an interior of the elongated cabin 100.

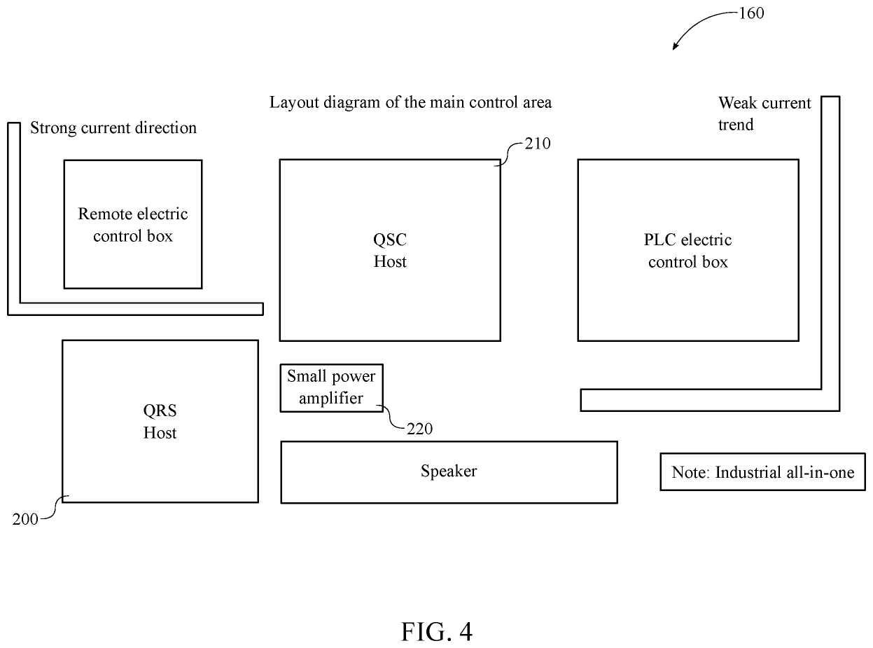

As can be seen in FIGS. 4, 5, and 7, the present invention may further comprise a QRS energy-pulse signal generator 200, a QSC full-spectrum frequency signal generator 210, and a signal amplifier 220. The QRS energy-pulse signal generator 200 is used to configure a pulse-based signal that is eventually outputted by the scalar-wave generating device 130. The QSC full-spectrum frequency signal generator 210 is used to configure a frequency-based signal that is eventually outputted by the at least one Tesla coil 140. The signal amplifier 220 is used to amplify the pulse-based signal so that the pulse-based signal can be effectively outputted by the scalar-wave generating device 130. Thus, the processing system 150, the QRS energy-pulse signal generator 200, the signal amplifier 220, and the scalar-wave generating device 130 need to be in serial electronic communication with each other, which allows the processing system 150 to trigger the creation of the pulse-based signal with the QRS energy-pulse signal generator 200, then allows the signal amplifier 220 to amplify the pulse-based signal, and finally allows the scalar-wave generating device 130 to output the pulse-based signal. The scalar-wave generating device 130 uses at least one scalar coil to output the pulse-based signal. The at least one scalar coil can be, but is not limited to, a quantum convert (QC) coil, which is a coil with its wire-intersecting nodes forming a straight line that is parallel to a central axis of the coil (e.g., a caduceus coil). A QC coil has a cancelling effect of the magnetic fields at its wire-intersecting nodes due to the opposing magnetic fields summing to zero.

As can be seen in FIGS. 4, 5, and 7, the signal amplifier 220 is also used to amplify the frequency-based signal so that the frequency-based signal can also be effectively outputted by the at least one Tesla coil 140. Thus, the processing system 150, the QSC full-spectrum frequency signal generator 210, the signal amplifier 220, and the at least one Tesla coil 140 need to be in serial electronic communication with each other, which allows the processing system 150 to trigger the creation of the frequency-based signal with the QSC full-spectrum frequency signal generator 210, then allows the signal amplifier 220 to amplify the frequency-based signal, and finally allows the at least one Tesla coil 140 to output the pulse-based signal. The at least one Tesla coil 140 can be, but is not limited to, a QC coil or a more traditional electromagnetic coil. The at least one Tesla coil 140 is preferably a plurality of Tesla coils that are positioned offset from each other. The plurality of Tesla coils is used to generate a stronger scalar wave with a stronger energy field and more energy information.

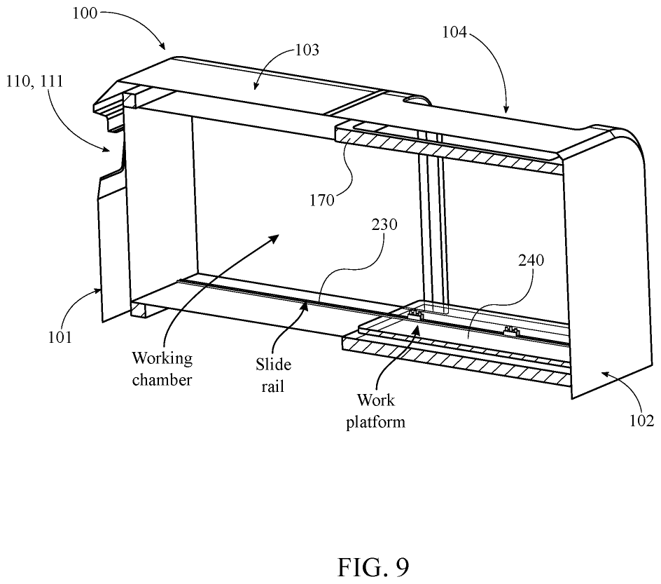

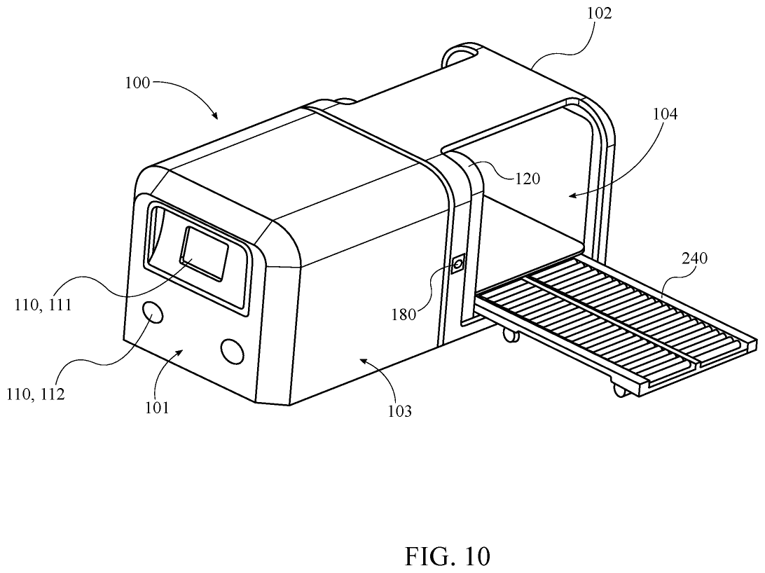

As can be seen in FIGS. 9 and 10, the present invention may further comprise at least one slide rail 230 and a work platform 240. The work platform 240 is used to situate an item within the elongated cabin 100 as analysis is conducted on the item by the present invention. The at least one slide rail 230 guides the work platform 240 along an internal length of the elongated cabin 100. The work platform 240 is moved along the at least one slide rail 230 because an item may need to be closer to the scalar-wave generating device 130 or closer to the at least one Tesla coil 140 depending on the kind of analysis being performed on the item. Thus, the at least one slide rail 230 and the work platform 240 are positioned within the elongated cabin 100. The at least one slide rail 230 is connected along a base of the lateral cabin portion 103 so that the at least one slide rail 230 allows the work platform 240 to move along the entire internal length of the elongated cabin 100. The work platform 240 is movably mounted along the at least one slide rail 230 so that the work platform 240 is able to slide to any position along the at least one slide rail 230.

### Supplemental Description

The device of the present invention comprises five major parts: a main cabin body, an operation display, an article slide sending device, a power supply circuit design, and a QC coil module. The main cabin body adopts thick sheet blister, ABS engineering plastic (i.e., including a shell, a cabin body door, various switches on the panel, an indicator light, etc.). The operation display allows a user to interact with an “Energy information frequency combination software” that is running in the Windows system, which can output eight different vibration frequency combinations. The operation display includes a panel, a power switch that controls the computer on/off, a QRS control switch that shows QRC working status/stop, a QSC control switch that shows QSC working status/stop, and a heat sink. The article slide sending device adopts a unique automatic article sending structure and is actuated by control buttons. The power supply circuit design includes a cabin operation of 50-Hertz (Hz) alternating current (AC) at 220 volts (V), a main engine direct current (DC) at 36 V that is approximately 1.5~2.5 Amperes (A), a power amplifier circuit module, a QRS host control, a QSC host controller, a power supply circuit module, a dedicated work indicator light, and an audio box part.

In terms of the QC coil module, “scalar wave frequency energy coil” is an important part of this product. Since the scalar energy does not radiate like an electromagnetic wave, it expands outward in an energy loop, filling the space to form a resonant energy system field. We use a special QC coil winding method (Tesla coil) to create a “scalar wave energy field” in the cabin. At the same time, the single-sided two-wire Tesla coil gains the resonance unit, which can be “planted” in the product without losing energy intensity “into” a set of curing frequencies.

Multi-dimensional wave frequency energy information implanting is a technology that generates scalar energy through a QC coil and loads it onto an object. The principle of the technology is that the weak current with frequency generates scalar waves through the QC coil, which resonates with the object but without changing the original properties of the object.

The QC geometric coil is used to generate a clockwise rotating field to transfer scalar energy and information. Multiple zero-point energy vortices produce a special harmonic interference, creating a clockwise rotating energy field around the coil, and this harmonic energy will be several times stronger than the original. In addition, there is a scalar wave coil on one side of the coil, which generates a gain electromagnetic field, it thus can push the scalar energy information wave frequency into the object.

In terms of scalar wave frequencies for energy information, through the research and practice of nearly five years, we have researched and developed the “Energy information frequency combination software” , and established a powerful, convenient, and fast energy information pulse signal (QRS) and frequency combination generation system (QSC). Eight kinds of different vibration frequency combinations have been designed and developed.

In terms of QRS energy information pulse signal transmission software, the software system has established nearly 2,000 health energy information codes, each code can generate a specific weak magnetic pulse signal, which is transmitted to the QC coil through the signal amplifier to form a scalar energy information field. In practical application, a number of energy information codes can be selected through the computer software to form a combination and act on the items in the cabin.

In terms of QSC frequency combination generation system, the present invention adopts the internationally standard full spectrum technology of Scalar Wave Frequencies, aiming at different types and characteristics of items, the program design has a total of more than 10,000 wave frequency spectra, with the lowest 0.5 Hz and the highest frequency 34,000 Hz. Given specific for implantable objects, wave frequency setting will be specified to the implanted object. The conventional setting must set the frequency value as: 7.83 Hz, 432 Hz, 528 Hz, 369 Hz, 417 Hz, and so on.

The software system sets the options of transmitting sine wave frequency and simulating scalar wave output and sets the modulation control of waveform selection. After the frequency combination is set, the frequency signal of the output-tuned sine wave is transmitted to the QC coil by the signal amplifier to form the scalar energy information field, which is used on the goods in the cabin.

It has been proved by experiments and related literature reports at home and abroad that the scalar wave technology and the torsion field technology independently studied in Russia for more than 50 years are essentially the same, both are vortices. By using the scalar wave resonance technique, the ultra-fine vibration wave which can change the physical characteristics of matter is developed and loaded into a specific material. The article material can achieve at least the following effects:
1. It can improve and enhance the quality of materials,
2. It can reduce and shield the impact of artificial frequency (60 Hz) on the human body.
3. The most important point is to adjust and balance human cells, so that the charge generated across the cell membrane reaches the best level of 70-90 millivolts, adjust the balance of the body's physical energy field, increase the whole body's energy level, accelerate the body's metabolism and function.
4. Spectroscopic analysis confirms that scalar waves increase the energy covalence level of each hydrogen atom in the body. This is important because hydrogen bonds are the bond that binds DNA together.
5. It can reduce the surface tension of the material and makes it easier for the body to absorb the edible product after it acts on the human body.

The multi-dimensional wave frequency energy information implanting compartment in FIG. 8 is a large rectangular-shaped shell. The operating unit control panel is at the front of the shell, and the placement unit compartment is at the rear. All kinds of industrial goods and agricultural products are placed through the sliding rail of the hatch door, such as food, health care products, etc, to the cabin inside.

The present invention is started with the power switch of the control panel, then selected the QRS energy information pulse signal or QSC frequency combination to generate transmission, and finally run the PC software, at the same time the indicator lights up. In addition, the Tesla coil with QC coil winding method is installed inside the main cabin body, and the scalar wave generator device (scalar coil) is installed at the tail of the cabin body, and the pulse signal and frequency signal are transmitted to the two groups of coils through the signal and frequency amplifier, and the fluctuation energy field is quickly generated. QC coil (Tesla coil) is the formation of a number of zero-point energy vortices, producing a special harmonic interference, and around the coil (cabin) produces clockwise rotation of the energy field, and this harmonic energy will be several times stronger than the original. In addition, there is a scalar wave coil at the rear of the cabin, which can generate a gain of the fluctuating energy field, therefore pushing the wave frequency of the scalar energy information into the object. Moreover, the scalar wave frequency generated in this process forms a multi-dimensional energy field. Through the principle of wave frequency resonance, the multi-dimensional energy field forms a resonance effect with the items in the cabin body, rapidly improving the energy value of items or food and the quality and efficacy of drugs and health products.

Although the invention has been explained in relation to its preferred embodiment, it is to be understood that many other possible modifications and variations can be made without departing from the spirit and scope of the invention as hereinafter claimed.

What is claimed is:

1. An implanting device using multidimensional wave frequency scalar wave-energy information comprising:
an elongated cabin;
a main control panel;
a hatch door;
a scalar-wave generating device;
at least one Tesla coil;
a processing system;
a power supply circuit;
the elongated cabin comprising a first cabin end, a second cabin end, a lateral cabin portion, and a hatchway;
the hatchway being integrated into the lateral cabin portion, adjacent to the second cabin end;
the hatch door being mounted across the hatchway;
the main control panel being externally mounted to the first cabin end;
the scalar-wave generating device and the at least one Tesla coil being mounted within the elongated cabin;
the scalar-wave generating device being positioned adjacent to the first cabin end;
the at least one Tesla coil being positioned offset from the first cabin end;
the main control panel, the scalar-wave generating device, and the at least one Tesla coil being electronically connected to the processing system; and
the control panel, the scalar-wave generating device, the at least one Tesla coil, and the processing system being electrically connected to the power supply circuit.

2. The implanting device using multidimensional wave frequency scalar wave-energy information as claimed in claim 1 further comprising:
an automatic door-sliding mechanism;
an electronic door control;
the hatch door being operatively mounted to the hatchway by the automatic door-sliding mechanism, wherein the automatic door-sliding mechanism is used to open and close the hatch door in order to provide internal access to the elongated cabin through the hatchway;
the electronic door control being externally mounted to the lateral cabin portion, adjacent to the hatchway;
the electronic door control being electronically connected to the automatic door-sliding mechanism; and
the automatic door-sliding mechanism and the electronic door control being electrically connected to the power supply circuit.

3. The implanting device using multidimensional wave frequency scalar wave-energy information as claimed in claim 1 further comprising:
the main control panel comprising a display screen and at least one control button;
the display screen being positioned offset from a base of the lateral cabin portion; and
the at least one control button being peripherally positioned around the display screen.

4. The implanting device using multidimensional wave frequency scalar wave-energy information as claimed in claim 1 further comprising:
a heat-sinking ventilation mechanism;
the heat-sinking ventilation mechanism being integrated into the first cabin end; and
the heat-sinking ventilation mechanism being positioned in between the main control panel and a base of the lateral cabin portion.

5. The implanting device using multidimensional wave frequency scalar wave-energy information as claimed in claim 1 further comprising:
a quantum resonance system (QRS) energy-pulse signal generator;
a signal amplifier; and
the processing system, the QRS energy-pulse signal generator, the signal amplifier, and the scalar-wave generating device being in serial electronic communication with each other.

6. The implanting device using multidimensional wave frequency scalar wave-energy information as claimed in claim 1 further comprising:
a quantum scalar convert (QSC) full-spectrum frequency signal generator;
a signal amplifier; and
the processing system, the QSC full-spectrum frequency signal generator, the signal amplifier, and the at least one Tesla coil being in serial electronic communication with each other.

7. The implanting device using multidimensional wave frequency scalar wave-energy information as claimed in claim 1 further comprising:
the at least one Tesla coil being a plurality of Tesla coils; and
the plurality of Tesla coils being positioned offset from each other.

8. The implanting device using multidimensional wave frequency scalar wave-energy information as claimed in claim 1 further comprising:
at least one slide rail;
a work platform;
the at least one slide rail and the work platform being positioned within the elongated cabin;
the at least one slide rail being connected along a base of the lateral cabin portion; and
the work platform being movably mounted along the at least one slide rail.
